(19) United States
(12) Patent Application Publication
(10) Pub. No.: US 2023/0304988 A1
(43) Pub. Date: Sep. 28, 2023

(54) METHOD AND APPARATUS FOR MEASURING WHITENESS AND/OR TESTING DYE AFFINITY OF FILAMENTS

(71) Applicant: LENZING AKTIENGESELLSCHAFT, 4860 Lenzing (AT)

(72) Inventors: Erich Kurzböck, 4690 Schwanenstadt (AT); Christoph Ramsauer, 5360 St. Wolfgang (AT); Christa Unterberger, 4863 Seewalchen (AT); Dominik Ostaszewski, 4690 Rüstorf (AT); Christoph Schrempf, 4701 Bad Schallerbach (AT)

(21) Appl. No.: 17/781,468

(22) PCT Filed: Dec. 1, 2020
(86) PCT No.: PCT/EP2020/084049

§ 371 Date: Jun. 14, 2023

(30) Foreign Application Priority Data
Dec. 2, 2019 (EP) 19212907.0

(51) Int. Cl. G01N 33/36; D06H 3/00; D03D 15/225; D03D 15/54

(52) U.S. Cl. CPC G01N 33/365; D06H 3/00; D03D 15/225; D03D 15/54

(57) ABSTRACT

The present invention relates to a method and apparatus for whiteness measurement and/or staining control of filaments.

### BACKGROUND OF THE INVENTION

### Field of the Invention

The present invention relates to a method and an apparatus for whiteness measurement and/or staining control of yarns and filaments.

### State of the Art

Yarns and filaments, especially based on cellulose, are produced on a large scale and used in many fields, such as textile industry but also in technical fields. An example of such filaments are filaments produced by the lyocell process from a composition of cellulose in a solvent, usually a mixture of water and N-methylmorpholine-N-oxide (NMNO). With regard to quality control of the filaments produced, the degree of whiteness as well as the dyeability are relevant in addition to mechanical characteristic values.

In the production of yarns, such as staple fiber yarns, filament yarns (monofilament and multifilament yarns), hereinafter referred to as yarns/filament yarns, in particular lyocell filament yarns, the yarns/filament yarns produced are wound onto bobbins after the spinning process and, if necessary, after optional post-treatments, made available for further use. Due to the high production speeds, several thousand such bobbins may be produced per day in a Lyocell plant, which then have to be fed to the post-treatment inspection for the desired properties. The results obtained should be as exact and reproducible as possible, especially with regard to the control of whiteness and dyeability. At the same time, the effort (labor input as well as space requirement of the devices) for these measurements should be as low as possible and preferably an essentially automated process should be realizable.

Currently, for whiteness measurement and/or staining (dyeability) control of yarns/filament yarns, in particular Lyocell filament yarns, test samples are knitted (knitted tubes), which are then used for whiteness measurement and/or staining control. Such methods are disclosed in WO 99/40428 A1, US 2009/190132 A1, EP 1 006 225 A2 and in Wehlow A. et al, Prüfverfahren der Textil- and Bekleidungstechnik, Springer, 2000, Chapter 8.1, pages 727 to 776 and Abdel-Fattah M Seyam et al, Fibers and Polymers, The Korean Fiber Society, Heidelberg, Vol. 13, No. 6, Aug. 1, 2012. These are still largely manual determinations, so that a variation of results due to the “human” influence factor is inevitable. In particular, it has been shown that the results of these checks can be erroneous, for example because the knitted samples tend to falsify the measured values during the measurements due to different mechanical loads (e.g. stretching of the sample). It is also difficult to compare such samples against a standard material. Furthermore, the respective knitting heads must be adapted to the titers of the filaments to be tested. This leads to downtimes due to the necessary exchange of knitting heads, and automation or continuous testing of a large number of samples is also not possible. Knitting machines are also comparatively susceptible to faults, which can again lead to downtimes. The space required for storing the samples is also comparatively high.

### Object of the Present Invention

It is therefore the underlying object of the present invention to provide a method and an apparatus that enables whiteness and/or staining control, preferably both, in filament yarns, in particular in lyocell filaments, overcoming the disadvantages of the prior art.

### BRIEF DESCRIPTION OF THE INVENTION

The present invention therefore provides a method according to claim 1 and an apparatus according to claim 8. Preferred embodiments are indicated in the subclaims and in the following description.

### BRIEF DESCRIPTION OF THE FIGURE

FIG. 1 schematically shows a process flow of the present invention or a schematic representation of an apparatus according to the invention.

### DETAILED DESCRIPTION OF THE INVENTION

Unexpectedly, it has been shown that the use of woven samples instead of knitted samples can overcome the disadvantages of the prior art. In the following, the invention is first described in detail with respect to the claimed method. However, it will be apparent to those skilled in the art that the following explanations also apply with respect to the claimed apparatus.

With regard to the yarns/filament yarns to be evaluated, the present invention is not limited to one type of yarn. Filament yarns, i.e., both monofilament and multifilament yarns can be tested, as well as other types of yarns, such as staple fiber yarns. For simplicity, these different types will be referred to hereinafter by the term yarn/filament yarn.
As already stated, the yarns/filament yarns are preferably Lyocell yarns/filament yarns. However, other cellulose-based yarns/filament yarns can also be used. Cellulosic yarns/filament yarns obtained by the viscose process, the cupro process or by regeneration from ionic liquids are also particularly suitable.

According to the invention, it is essential that a woven sample is used for the whiteness and/or staining control. By using a woven sample, the control of the properties relevant here in particular do not suffer from the fluctuations and irregularities of the measurements that occur due to the lower mechanical strength of the knitted samples in the prior art. At the same time, the devices to be used for producing the woven samples, preferably ribbon looms, are less susceptible to faults than knitting machines, so that a larger number of bobbins of yarn/filament yarn can be tested per unit of time.

In the production of the woven samples, which may be given the shape of a rectangular fabric, for example, the yarns/filament yarns to be tested are preferably used as weft yarns. Standard filament yarns of a precisely defined titer and a known whiteness are preferably used as warp yarns (warp threads). These warp yarns can be Lyocell yarns/filament yarns, but the use of warp yarns of other materials is also possible and in embodiments even preferred. For example, other natural yarns or synthetic yarns are suitable. An example of a synthetic yarn are polyester yarns, which are readily commercially available in consistent, standardized quality. By using such warp threads, due to the higher strength of these materials, the strength of the woven sample can again be increased, further increasing the advantage of using a woven sample (compared to knitted samples). The warp threads can be monofilament or multifilament, and all other types of yarns and twists are also suitable. Since such ribbon weaving machines can produce fabrics of the same quality regardless of the titer of the weft yarns (for example, by adjusting the number of wefts, something that can be done automatically during the production of the samples, taking into account the titer of the filament to be evaluated), there is also no need for special adaptation of the ribbon weaving machine as such to different types of yarn/filament yarn (titer). This means that samples can be produced from different filament yarn types without any great effort in terms of equipment (no conversion work is necessary, unlike with knitting machines, where it is necessary to replace the knitting heads depending on the titer of the filament yarns to be tested).

Since the introduction of filament yarns into such ribbon looms is possible through the use of commercially available yarn feeders, which either automatically connect the beginning of each new filament yarn (typically from a bobbin) to the end of the previously fed filament yarn, or directly engage and subsequently fix the beginning of a new filament yarn to the weaving needle by an automatic device, sample specimens (for example, woven specimens of about 6×10 cm) of yarns/filament yarns from different bobbins can thus be continuously produced. By appropriately controlling, for example, the pausing of the weaving needle, which results in filament yarn interconnections between the individual woven sample pieces, it is easy to distinguish between the individual woven samples. This woven construction further forms a connection between the respective beginning or end of the specimen samples to be tested, so that with regard to the control of the degree of whiteness and/or the dyeability, which will still be done, a continuous and also automated process control remains possible.

As already explained, a suitable yarn, preferably monofilament or multifilament yarn, can be used as warp yarn. This can be prepared for weaving, for example by providing a sizing or by preparation with an oil. In particular, twisted, intermingled, or flamed yarns/filament yarns are suitable. Yarns/filament yarns of all these variants with a titer of 60 to 160 dtex, preferably 80 to 120 dtex, are particularly suitable. These warp yarns, with the same weave (weaving conditions), have only a negligible influence on the properties relevant and to be determined here. The yarn/filament yam to be tested is used as weft yarn, so that the woven sample piece will have a dominant, weft-affected side on which the relevant measurements can then be carried out. By adjusting the weft density, yarns/filament yarns of different titers can also be processed to produce comparable woven samples. In general, the weft density is increased for finer titers, in particular to ensure sufficiently high fabric strength values and weft densities as a measuring surface. Exemplary combinations of titers of the yarns/filament yarns to be evaluated and weft density are as follows: (titer weft/weft density) 40 dtex/26 weft per cm; 500 dtex/10 weft per cm (when using a warp beam with 25 warp threads per cm and atlas weave 3/1).

It has been shown that in this way yarns/filament yarns with different titers (weft thicknesses), for example from 10 to 500 dtex, can be processed without problems into fabric samples for subsequent whiteness determination and/or staining control.

Thus, by using woven samples, it is already possible to overcome several of the disadvantages encountered in the prior art. For example, specimens can be produced from bobbins in an automated and continuous manner, since commercially available tape looms and yarn feeders make automated and continuous specimen production feasible. These systems are also less prone to malfunction than knitting machines, and downtime due to the need to replace the knitting head (to control bobbins with different filament yarn titers), if necessary, during the production of knitted samples is eliminated. At the same time, the linking or automated direct yarn/filament yam addition, in the case of woven samples, simplifies further continuous evaluation without the need to manually create links/connections for this purpose, for example. Compared to the production of knitted samples, the production of woven samples is also faster, which increases the possible bobbin throughput. In addition, the woven samples are less mechanically susceptible, so that measurements of whiteness and/or dyeability are less prone to errors/deviations. Furthermore, by using a yarn/filament yarn with known properties as the warp yarn, this can theoretically also be considered as an internal standard in the woven samples and used accordingly in subsequent evaluations.

The samples prepared as described above can then be submitted for further evaluation, individually and manually, but preferably automated and continuously.

One evaluation to be performed according to the invention is the determination of the whiteness. As already described above, this determination is carried out on the dominant weft side. This makes the influence of the warp threads used irrelevant for the measurement. The conditions and apparatus necessary for determining the degree of whiteness are known to the skilled person. Known, commercially available measuring apparatuses can be used.

Another evaluation to be carried out according to the invention is a dyeing (staining) control of the yarns/filament yarns. For this purpose, the woven sample is dyed by a standardized method and then the color value is determined. In this process, the fabric is usually first subjected to a dye application in a dyeing foulard, followed by fixation, for example in a steamer, washing and drying. Suitable dyes are in particular direct dyes, such as solophenyl blue. Essential with regard to the evaluation and comparability of the measurement results are constant dyeing conditions, which is known to the skilled person, as well as the conditions during the individual steps (such as dyeing, fixing, washing, drying). The method according to the invention, which can be run continuously, makes it possible to minimize the use of chemicals in the staining control, in comparison with the non-continuous staining controls with knitted samples.

After completion of the dyeing process, the color value is determined. Again, this is done on the dominant weft side of the fabric, so that the influence of the warp thread on the result of the determination is again negligible. The necessary conditions and equipment are also known to the skilled person for this purpose. Known and commercially available measuring apparatuses can also be used.

According to the invention, the method according to claim 1 is designed such that the whiteness of the yarn/filament yarn is determined in a whiteness measuring station on the dominant weft-accentuated side of the woven sample piece and/or the color value of the yarn/filament yarn is determined after dyeing of the woven sample piece on the dominant weft-accentuated side of the woven sample piece.

According to the invention, both a determination of the degree of whiteness and a determination of the color value are preferably carried out in this sequence. Due to the connection between the individual woven test pieces already described above, these can in turn be continuously and automatically guided through the individual steps of determining the degree of whiteness and the color value.

After the determination of the color value, the samples can be fed to storage. For this purpose, they can be wound up, for example, so that rolls with the individual samples, which are preferably still connected, are obtained. Such rolls can be stored easily and with a low space requirement, a further advantage over the use of knitted samples, since there the space requirement for storage is greater and also associated with higher manual effort.

Since, on the one hand, the production of the woven test pieces and, on the other hand, the determination of the degree of whiteness as well as the dyeing and color value determination take different amounts of time, in embodiments the test pieces are temporarily stored between the aforementioned steps. Due to the normally higher time requirement of the dyeing step, compared to the weaving of the sample pieces as well as the whiteness determination and color value measurement, intermediate storage of the woven sample pieces, especially before the dyeing step, is helpful in many embodiments. In continuous processes, such intermediate storage can also be achieved by adjusting the length of the links (the link between the filament yarn end and filament yarn beginning of two filament yarns originating, for example, from bobbins to be tested in succession) between the individual woven test pieces. In this way, a largely automated and continuous process can be realized. In this process, the interconnected woven sample pieces are continuously fed to the individual steps of the process by suitable transport systems.

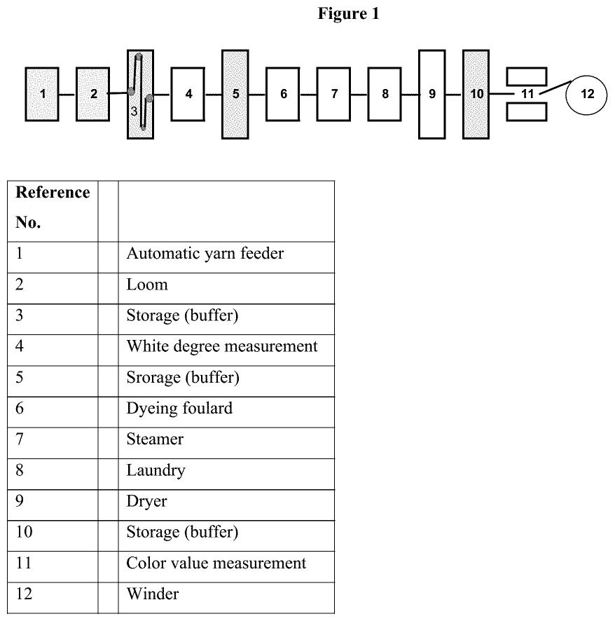

Thus, with reference to FIG. 1, a method according to the invention may comprise the following steps:
1.) The bobbins with filament yarn coming from the filament production (i.e., for example, from the winding machine) are introduced one after the other into the automatic yarn feeder (1). The beginning of a new filament (from a new bobbin) can be introduced into the weaving machine by this and thereby automatically linked to the end of the filament of the previous bobbin. Alternatively, any yarn/filament can be inserted fresh directly (see also [0012]).
2.) A sufficient length of filament yarn is drawn from the bobbin and woven into a sample piece in a weaving machine (2), for example a ribbon loom. The filament yarn to be tested is used as the weft thread.
3.) After a sufficiently large sample piece has been woven (for example with a width of about 10 cm and a length of 6 cm) and a sufficiently long end piece of the filament yarn has additionally been drawn off (for possible linking with the filament yarn of the next bobbin and to control/enable the optional intermediate storage times, for example in a store (3) between the individual, subsequent further steps), the filament yarn is cut so that the bobbin can be packed, stored and made available for further use.
4.) The woven sample piece is now fed to the whiteness measurement (4) and then optional intermediate storage can again take place in a further storage unit (5).
5.) The sample is then dyed, for example in a dyeing foulard (6), followed by a steam treatment (7) to fix the dyeing, a washing stage (8) and drying (9). Optionally, prior to the subsequent determination of the color value (11), the sample can be stored again in a storage unit (10).
6.) After going through the steps described above, the test pieces can be wound up by a winder (12) and then stored, for example in the form of a roll.

In the process according to the invention, the relevant data, i.e. in particular bobbin number (product information on the filament or filament yarn, such as titer, etc.), weaving parameters (number of wefts but also type and titer of the warp thread), result of the whiteness measurement, parameters of the dyeing, result of the color value determination, day of the production of the sample piece, etc., are preferably stored automatically by suitable processes, so that a data record with the relevant information is available for each evaluated bobbin. This can be done by automatically recording the operating parameters of the weaving machine, the dyeing foulard, etc., and by correspondingly recording the measurement results. The systems required for this are known to the skilled person. This data can then also be fixed in whole or in part in the form of a label on the sample piece itself, so that direct identification of the sample pieces is also possible. Labels can be suitably attached to the sample (glued, tacked, etc.) or printed directly on the sample, for example as a bar code. The necessary devices etc. are known to the skilled person.

This generates a complete data set of relevant information for each bobbin, allowing storage of relevant product parameters via unique identification of the sample piece and bobbin.

According to the invention, in addition to the method described above, an apparatus for the continuous and preferably automated production and evaluation of yarns/filament yarns is also provided. This device comprises at least one weaving machine for producing a woven sample piece and at least one unit for determining the whiteness of the sample piece or a unit for dyeing and determining the color value of the sample piece. Preferably, these devices are connected by suitable means which allow continuous and largely automated guidance of a plurality of optionally interconnected sample pieces through the device.

Thus, with reference to FIG. 1, such a device may include the following components:

A.) An automatic yarn feeder (1). This feeds the beginning of a filament yarn of a bobbin into a weaving machine (2) where this beginning is optionally automatically linked to the end of the filament yarn of the previous bobbin. Alternative embodiments are possible as described in paragraph [0012]. For this purpose, an automatic insertion machine is then used, for example. The yarn/filament yarn to be evaluated is inserted into the weaving machine as a weft yarn and processed into a woven sample piece.

B.) After the loom, a storage (3) can be provided. Then a whiteness meter (4) is provided, followed again by an optional storage (5).

C.) A dyeing foulard (6) can then be provided, followed by a steamer (7) for fixing the dyeing, a washer (8) and a dryer (9). Optionally, a new storage unit (10) can then be provided before the subsequent unit for color value determination (11).

D.) Finally, a unit for producing a compact storage form of a plurality of specimens may be provided, such as a winder (12) that rolls up the specimens into an easily storable roll of retention samples. Not shown in FIG. 1 is the unit necessary for continuous and automated process control for continuous and controlled guiding of the test pieces through the device. However, such devices are known in particular to the skilled person in the field of filament yarn production and weaving machines, for example roller and reel systems.

The process according to the invention and the apparatus according to the invention can therefore continuously and automatically process yarn/filament yarn samples into test pieces and then subject them to a whiteness measurement and/or color value determination after dyeing, so that relevant yarn/filament yarn data can be obtained in a reproducible manner. The space requirement of the device is lower compared with methods using knitted test pieces, and the throughput of bobbins is also significantly higher according to the invention (no replacement of knitting heads, the system as a whole is more robust and less susceptible to faults due to the use of a weaving machine). The data obtained by the method according to the invention are subject to lower errors with regard to the yarns/filament yarns to be measured/evaluated, since the woven test pieces are less extensible, the titer of the yarn/filament yarn has no influence on the determinations of whiteness and color value due to the use of a woven test piece. Also, the space requirements for storing the reserve samples are less, so that costs can be saved here as well. Likewise, the automatic and continuous recording of the relevant data can significantly reduce the scattering of results that occurs when measurements are carried out manually and in batches.

Furthermore, the continuous dyeing of the woven test pieces can significantly reduce the material input and energy consumption in these steps compared to discontinuous processes, especially with knitted test pieces.

Overall, therefore, a significantly improved system for collecting relevant data for yarns/filament yarns, in particular lyocell filament yarns, is provided.

1. A method for determining a degree of a whiteness and/or a color value of a cellulosic yarn/filament yarn, optionally a lyocell filament yarn, wherein a woven sample piece is produced from the cellulosic yarn/filament yarn to be evaluated, the cellulosic yarn/filament yarn to be evaluated being used as a weft yarn, and wherein the whiteness is determined on a dominant weft-accentuated side of the woven sample piece and/or the color value of the cellulosic yarn/filament yarn, after dyeing of the woven sample piece, is determined on the dominant weft-accentuated side of the woven sample piece.

2. The method according to claim 1, wherein the lyocell filament yarn or a synthetic fiber having a titer of 60 to 160 dtex is used as a warp yarn.

3. The method according to claim 1, wherein after production of the woven sample piece, first the whiteness is determined and then, after dyeing of the woven sample piece, the color value is determined.

4. The method according to claim 1, wherein different cellulosic yarns/filament yarns are successively processed in a continuous process to produce the woven sample piece and the woven sample piece is continuously subjected to determining the degree of the whiteness and the color value, after dyeing of the woven sample piece.

5. The method of claim 4, wherein a plurality of the woven sample piece is joined together by non-woven warp sections.

6. The method according to claim 4, wherein a plurality of the woven sample piece is wound up into a roll after determining the whiteness and the color value.

7. The method according to claim 1, wherein the dyeing is performed by using a direct dye.

8. An apparatus for determining a degree of a whiteness and/or a color value of a cellulosic yarn/filament yarn, optionally a lyocell filament yarn, comprising a weaving machine in which the cellulosic yarn/filament yarn to be evaluated is processed into a woven sample piece, and a unit for determining the degree of the whiteness and/or a unit for determining the color value of the cellulosic yarn/filament yarn on the basis of the woven sample piece.

9. The apparatus of claim 8, further comprising an automatic yarn feeder, for introducing the cellulosic yarn/filament yarn into a loom.

10. The apparatus according to claim 8, wherein the unit for determining the color value further comprises a dyeing foulard, a steam unit for fixing, a washer and a dryer.

11. The apparatus according to claim 8, further comprising a unit for continuously guiding a plurality of the woven sample test piece through the apparatus.

12. The apparatus according to claim 8, further comprising a system for automated acquisition of data from the loom, the unit for determining the degree of the whiteness, and the unit for determining the color value.
